(19) United States
(12) Patent Application Publication
(10) Pub. No.: US 2014/0276178 A1
(43) Pub. Date: Sep. 18, 2014

(54) Multi-lumen breathing tube device

(71) Applicant: David Lew Simon, Mansfield Center, CT (US)

(72) Inventor: David Lew Simon, Mansfield Center, CT (US)

(21) Appl. No.: 13/815,623

(22) Filed: Mar. 13, 2013

(51) Int. Cl. A61M 16/04; A61M 16/20; A61M 16/00; A61M 16/22; A61M 16/08; A61B 5/097

(52) U.S. Cl. CPC A61M 16/0434; A61M 16/0816; A61M 16/0875; A61B 5/097; A61M 16/0078; A61M 16/22; A61M 16/208
USPC 600/543; 128/207.14; 128/207.15

(57) ABSTRACT

A multi-lumen breathing tube device providing for separate and bidirectional gas flow for expired and inspired gas during ventilation of a lung. Embodiments of the multi-lumen breathing tube include an improved laryngeal mask airway device and an improved endotracheal tube. The invention is compatible with a circle breathing circuit system used with many mechanical ventilators including those used in conjunction with anesthesia machines.

### TECHNICAL FIELD

The present invention relates to breathing tubes used for ventilation of the lungs, as during anesthesia, or as required in critically ill patients. More particularly, tubes and tube connectors and adapters are disclosed that relate to endotracheal tubes, or to supraglottic airway devices such as a laryngeal mask airway. The invention provides for improved ventilation with endotracheal tubes and supraglottic airway devices that are used in conjunction with a typical breathing circuit. Also disclosed is an improved laryngeal mask component of a supraglottic airway device.

More specifically, the invention relates to a multi-lumen tube device that allows for compartmentalized bi-directional ventilation to and from the lungs of a living organism such as a mammal, or more specifically a human. Here “multi-lumen” is taken to mean at least two lumens. The invention allows for new air to be directed to or distal to the glottis region of a mammal.

### BACKGROUND (PRIOR) ART

Breathing tubes of various kinds are employed in the medical arts to assist with ventilation of the lungs. Ventilation is the process whereby respiratory gases, such as medicinal air (comprising oxygen and nitrogen), oxygen, and carbon dioxide, are delivered to and taken away from the lungs by inspiration and expiration (exhalation), respectively.

Endotracheal tubes are commonly employed to assist with ventilation. An endotracheal tube is a tube that is inserted through a patient's vocal cords into the trachea at one end, and interfacing with a flow of respiratory gas at the other end, thereby providing a means of ventilation to the lungs.

A supraglottic airway device is a device that is inserted to rest above the glottis, which is the middle part of the larynx where the vocal cords are located. Therefore, a supraglottic device does not go through the vocal cords into the trachea, but instead rests above the vocal cords to provide a means of ventilation to the lungs. A supraglottic airway device functions to provide an unimpeded path of ventilation past the upper airway to the opening of the vocal cords. For example, U.S. Pat. No. 7,159,589 B2 to Brain describes generally a laryngeal mask airway apparatus with an inflatable cuff. There are a number of supraglottic airway devices presently in the marketplace. Such examples of supraglottic airways are the laryngeal mask airway and its variants, such as the LMA Clasic Excel™, the LMA Supreme™, the LMA Unique™, the LMA Fastrach™, the LMA ProSeal™, the LMA Classic™, and the LMA Flexible™ manufactured by LMA, Inc. Other examples are Ambu® Aura40™ Reusable Laryngeal Mask, Ambu® AuraFlex™ Disposable Laryngeal Mask, Ambu®AuraOnce™ Disposable Laryngeal Mask, and the Ambu® AuraStraight™ Disposable Laryngeal Mask, manufactured by Ambu, Inc. Additional examples of supraglottic airway devices are the i-gel brand of supraglottic airways manufactured by Intersurgical Ltd.

Some of the aforementioned supraglottic and laryngeal mask airway devices include an additional lumen or tube that when inserted into a patient is introduced at its distal end to the opening of the esophagus. This provides for a means of draining fluid from the esophagus or stomach through the esophagus and distal end of the lumen to the proximal end of the supraglottic or laryngeal mask airway device so that the gastrointestinal fluid can be evacuated. Some embodiments are designed such that a Salem Sump™ or other flexible gastric tube can be passed through the esophageal lumen of the supraglottic or laryngeal mask airway device. U.S. Pat. No. 7,305,985 B2 to Brain describes a laryngeal mask airway with a second lumen for drainage of the oesophagus. The second lumen described therein cannot provide for passage of respiratory gases to and from the lungs, and does not teach the present invention. These esophageal or gastric lumens should not be confused with the compartmentalized breathing tube of the present invention. A lumen of the compartmentalized breathing tube of the present invention has a different form, structure and functionality as does the aforementioned esophageal or gastric tubes or lumens.

U.S. Pat. No. 7,546,838 B2 to Lin describes construction of a laryngeal mask airway. U.S. Pat. No. 7,047,973 B2 (“'973”) to Chang teaches a laryngeal mask airway with two ribs formed in the inflatable mask portion to prevent blockage of the breathing tube, and other improvements to avoid breakage and eliminate discomfort. Although '973 loosely uses the term “dual-airway tube,” this is not descriptive or suggestive of the present invention. '973 teaches that its dual airway tube has a primary tube that is large bore for transporting respiratory gases, and a secondary tube formed inside the wall of the primary tube that is too small in diameter to effectively transport respiratory gases to and from the lungs of a patient. Further, this secondary tube does not communicate with either the patient's lungs, or a fresh flow of respiratory gases for ventilation. Therefore, the term “dual-airway tube” of '973 should not be confused with the bi-directional breathing tube disclosed in the invention at hand; they are not similar in function, location, or size. U.S. Pub. No. 2006/0307601 A1 to Nasir describes an airway device comprising an airway tube, the distal end of which is surrounded by a non-inflatable pre-formed laryngeal cuff. None of these previous art references disclose the present invention.

There exists in the marketplace so-called “double lumen tubes” used to segregate air flow to different parts of the lung. A review of current technology relating to breathing tubes and airway devices is found in an article titled Supraglottic Airway Devices Other Than Laryngeal Mask Airway And Its Prototypes, by Doctors Sinha and Mistra in Indian Journal of Anaesthesia 2005; 49 (4), pages 281-292. See especially, page 284 under section J., Double lumen tubes, to distinguish between the double lumen tube (“DLT”) described therein and the present invention. The prior art DLT does not provide separate means of gas flow for inspiration and expiration. Rather, the prior art DLT merely provides both inspiratory and expiratory gas flow through the same lumen to different parts of the lungs. The two separate lumens of the DLT do not provide a means for separately ventilating inspiratory and expiratory gases. There does not presently exist in the marketplace a breathing tube as disclosed in the present invention.

Laryngeal mask airways as previously described comprise a laryngeal mask component that is either filled with air (see the “inflatable cuff” described in U.S. Pat. No. 7,159,589) or is made of a gel material (see U.S. Pat. Applic. No. 2006-0207601-A1). The air filled mask is adjustable in that the mask may be inflated or deflated by applying various amounts of air to the cuff, however it does not tend to naturally conform to the anatomy of the airway in which it sits. The “i-gel” laryngeal mask does tend to naturally conform to airway anatomy, but its fit cannot be manually adjusted. These are important limitations of the presently available laryngeal mask airways because the utility of the laryngeal mask airway is dependent upon a proper fit to the airway anatomy.

### SUMMARY OF INVENTION

### Technical Problem

The technical problem with the endotracheal tubes and supraglottic airways currently on the market is that in common usage they inefficiently provide for ventilation such that a portion of expired respiratory gas is re-inspired. Common usage for endotracheal tubes and supraglottic airway devices is in conjunction with a circle type breathing circuit. A circle breathing circuit is presented in FIG. 1. FIG. 1 demonstrates how a respiratory gas would be transported from the inlet for the source of gas flow 10, through one-way valve 5 in the inspiratory limb of the circuit, through corrugated tubing 3 of the inspiriatory limb, to a Y-piece 2, which may be connected to an “elbow” or right angle connector 1, and then to a breathing tube connector or to a breathing tube. Commonly the source of gas flow is a mechanical ventilator.

The gas then enters the patients lung where respiratory gas exchange occurs, and is expired. The expired gas passes out of the breathing tube, through the elbow 1, through the Y-piece 2, through the corrugated tubing of the expiratory limb of the breathing circuit 4, through a one-way valve 6 in the expiratory limb of the breathing circuit, passed a valve known as an “APL” valve 7, passed a reservoir air bag 8, and then is generally returned back to the circle circuit through a carbon dioxide absorber 9. Some of the expired gas may be diverted through a valve if the breathing circuit pressure exceeds a determined limit. Some of the gas may be siphoned off into a scavenger vacuum. However, the general principle is that a respiratory gas such as oxygen, nitrogen or carbon dioxide circles within the circuit, with carbon dioxide being removed from the circle by the carbon dioxide absorber 9, and oxygen, helium, nitrogen, and/or other medical gas being added as needed at the inlet for the source of gas flow 10, perhaps delivered by a mechanical ventilator. Because of the one-way valves in the inspiratory and expiratory limbs in the circle circuit, gas flow is unidirectional throughout the circle breathing circuit except at points distal to the Y-piece 2.

Without the present invention a breathing tube device generally consists of a single lumen, and gas flows in both directions through that lumen, in one direction during inspiration and in the opposite direction during expiration. Because the prior art breathing tube device is essentially composed of a single tube breathing tube, gas from inspiration may mix with gas of expiration at points distal to the Y-piece, and in the Y-piece itself 2, in the elbow 1, and in the breathing tube that may be part of a laryngeal mask airway device or which may represent an endotracheal tube. With the present invention, however, the inspiratory and expiratory gases do not mix in the Y-piece, the elbow, or the breathing tube.

The problem of re-inspired expiratory gas is troublesome for several reasons. First, the oxygen concentration in expired gas is lower than in inspired gas. Therefore, less oxygen can be administered when respiratory gas is re-inspired. Second, expiratory gas contains carbon dioxide. The carbon dioxide further dilutes the oxygen, thus providing a second mechanism for decreased oxygen concentration in re-inspired expiratory gas. Third, stagnant expiratory gas contains carbon dioxide whereas fresh un-re-inspired inspiratory gas would contain no or negligible amounts of carbon dioxide. Inspiring stagnant expired gas results in the patient inspiring carbon dioxide. This is not desirable because it results in carbon dioxide build-up in the patient, which can cause serious negative physiological effects.

In some cases the decreased inspired concentration of oxygen and inspiration of carbon dioxide may not lead to noticeable deleterious effects. Healthy adults may be able to tolerate this, which is why the current technology has been effective for most patients receiving artificial ventilation. However, there are certain patients that are more susceptible to the decreased oxygen and increased carbon dioxide who would greatly benefit from the present invention. Also, even healthy patients when breathing spontaneously during sleep may benefit from the present invention because in this situation breath volume (tidal volume) may be reduced compared to volumes of artificial ventilation as delivered by a ventilator machine.

Many patients suffer from severe lung disease who have marginal pulmonary reserve capacity. Some critically ill patients suffer from such lung pathology that it is very difficult to maintain oxygenation, even when given the maximum amount of oxygen that a ventilator will deliver at normal barometric pressure. For these patients the relatively small decrease in oxygen from dilution due to inspired exhaled respiratory gases can be clinically significant. Other patients are very sensitive to carbon dioxide. For example, patients with acute brain trauma are hyper-sensitive to alterations in carbon dioxide. Increasing carbon dioxide in these patients can cause cerebral blood vessel vasodilation with resultant increased intracranial pressure. These changes in intracranial pressure due to hypercapnia and hypercarbia can be life threatening. Thus, these patients would benefit from not re-inspiring expired respiratory gases.

The smaller a particular patient is, and the smaller the lung volume ventilated for that patient is, the greater are relative effects of inspiring a given volume of re-inspired expiratory gases. Therefore, the present invention would be of significant benefit to small patients. The amount of stagnant gases contained within breathing tubes currently available in the marketplace are clinically significant in very small patients. In such very small patients methods of ventilation alternative to the circle type breathing circuit are often sought. However, the circle breathing circuit is the standard type of circuit most often used in operating rooms and intensive care units. To date there is no readily available endotracheal tube or supraglottic airway device that does away with the stagnant gases as disclosed herein that is readily adapted for use with a circle type breathing circuit.

FIG. 2 demonstrates the problem of re-inspired expiratory gas due to stagnant gas flow. In FIG. 2 the vertical lines represent volume within the ventilation system that is filled with fresh gas from a source of fresh gas flow (the source being shown in FIG. 1). The horizontal lines in FIG. 2 represent volume that is filled with gas expired from the patient and delivered back into the ventilator circuit.

FIG. 2A through FIG. 2D represent the current problem with breathing tube assemblies available in the marketplace today. In FIG. 2A the inspiratory limb of the Y-piece 20, the expiratory limb of the Y-piece 22, and the breathing tube 26, along with the internal volume of the connector 24 that connects the Y-piece to the breathing tube 26, are filled with fresh gas. As an example, the fresh gas may be 21% oxygen and 79% nitrogen. This is the state immediately prior to the patient's first inspiration—prior to the first breath in—without the present invention. The entire volume is filled with fresh gas (21% oxygen and 79% nitrogen), represented by vertical lines. The patient then inspires (breaths in), respiratory gas exchange occurs in the lungs, and next the patients expires.

FIG. 2B represents the state immediately after the first expiration (after the patient breathes out). Now the situation is different. The breathing tube is filled with expired gas up to the region of the Y-piece that I term the “most distal point of fresh gas flow” in the breathing circuit (hereafter, “MDPFGF”), at 28. The inspiratory limb of the Y-piece 20 in FIG. 2B is filled with fresh gas (vertical lines) which is constantly flushed through the circle circuit, and the expiratory limb of the Y-piece 22 now contains a mixture of the fresh gas (vertical lines) flushing through the circuit and the gas just expired (horizontal lines).

FIG. 2C represents a short moment later (perhaps several seconds later, depending upon the respiratory rate and the rate of fresh gas flow), where the volume proximal to the MDPFGF 28 contains fresh gas (vertical lines) and the volume distal to the MDPFGF 28 contains expired gas (horizontal lines). This volume of expired gas is a stagnant volume of gas because it is beyond the MDPFGF 28. This stagnant gas would not contain the 21% oxygen and 79% nitrogen of fresh gas. Because some oxygen is utilized for respiratory gas exchange in the lungs, the percent of oxygen distal to MDPFGF in FIG. 2C is less, typically approximately 16 to 17 percent. Further, because carbon dioxide is a product of respiratory gas exchange in the lungs, the stagnant gas would also contain carbon dioxide. In a normal metabolic state the expired gas would contain an amount of carbon dioxide to exert a pressure of about 40 mm Hg, or about 5% of the expired gas at normal sea level barometric pressure. Thus, the stagnant gas would contain about 16% oxygen, 5% carbon dioxide, with the remaining 79% composed of nitrogen.

FIG. 2D represents the state immediately after subsequent expirations, which is essentially the same situation as in FIG. 2B. Thus a volume of stagnant gases containing 15% oxygen and 5% carbon dioxide will replace a like volume of what would have otherwise been a fresh gas mixture of 21% oxygen and no carbon dioxide. In large healthy patients, this stagnant volume may be so proportionally small as compared to the patient's tidal volume that is may have no deleterious effect on the patients. However, in sufficiently small patients the amount of stagnant volume may be a significant proportion of the tidal volume such that the extra carbon dioxide in the stagnant gas could result in carbon dioxide build up in the patient. Also, if the patient has sufficiently small tidal volumes, or if oxygen gas exchange is sufficiently impaired due to pathology of the lung or respiratory system, then there may be difficulty in oxygenation that would be deleteriously exacerbated by delivering only 16-17% oxygen in the stagnant volume instead of the usual 21%. In patients with very marginal capacities of oxygen exchange due to pathology it may be difficult to maintain oxygenation even with delivery of 100% oxygen. In these patients, a decreased in inspired oxygen due to the presence of carbon dioxide could decreased stagnant oxygen from 100% to 95% or less (depending on the carbon dioxide level). This change could have a deleterious effect on such patients.

Another technical problem with the supraglottic airways currently on the market relating to laryngeal mask airways is that it can be difficult to fit the laryngeal mask component to a particular patient's airway anatomy. If the fit is not snug enough, there will be leakage around the mask component, which leads to suboptimal ventilation, and can also fail to provide a barrier between the airway and the esophagus. Failure to provide such a barrier can result in gastric contents leaking into the glottis and lungs, which may result in deleterious lung pathology. If the fit is too tight, this can lead to trauma of the airway surrounding the laryngeal mask component. Such trauma may cause patient discomfort, and can also cause a swelling of the airway that could impead air flow after the supraglottic airway is removed. Thus, the optimal mask component will tend to naturally conform to a particular patient's airway, but also allow for manual adjustment as may be need in individual instances.

### Solution to Problem(s)

An inventive step of the present invention is providing for separate means of transporting inspiratory gas flow and expiratory gas flow by compartmentalizing the breathing tube into distinct lumens such that throughout most or all of the length of the breathing tube inspiratory flow is limited to one lumen and expiratory flow to another lumen. FIG. 1 demonstrates that gas flow through a circle type breathing circuit is unidirectional due to the presence of one-way valves on the inspiratory and expiratory limbs of the breathing circuit. FIG. 2E through FIG. 2H demonstrate how the present invention is an improvement over current technology.

FIG. 2E shows isolation of inspiratory gas flow from expiratory gas flow by inclusion of a septum 31 into the breathing tube 27, thus segregating the tube into two compartments or lumen—an inspiratory lumen 33 and an expiratory lumen 35. This effectively moves the MDPFGF from region 28 as shown in FIG. 2A to region 29 as shown in FIG. 2E. FIG. 2E through FIG. 2H demonstrate that the present invention eliminates the problem of stagnant gas in the breathing tube and results in a delivery of fresh gas flow (vertical lines) at the most distal end of the breathing tube in each stage of the ventilation cycle. FIG. 2E is immediately prior to the first inspiration (same stage of ventilation cycle as FIG. 2A). FIG. 2F is immediately after the first expiration (same stage of ventilation cycle as FIG. 2B). FIG. 2G is immediately prior to subsequent inspirations (same stage of ventilation cycle as FIG. 2C). FIG. 2H is at the same stage of the ventilation cycle as FIG. 2D.

The invention allows the new bi-directional or multi-lumen breathing tube to be used with conventional circle breathing circuit systems. Thus, the present invention also encompasses a breathing tube device allowing for inspiratory gas to be directed in one channel or lumen of the breathing tube, and allowing for expiratory gas to be directed in another channel or lumen of the breathing tube. This device may include connectors from the circle breathing circuit to the new breathing tube that act to keep the inspiratory and expiratory flows separated from one another. For example, a novel Y-piece is described, as well as a novel elbow or right angle connector, as well as a novel connector to connect the breathing tube to either the Y-piece or the elbow (i.e., the breathing tube connector). In addition, the device may contain components that make up a laryngeal mask airway device or cuffed endotracheal tube.

Another inventive step disclosed herein is to provide for a laryngeal mask component comprising a memory foam surrounded by a plastic material that is impermeable to ventilator gasses, such that the mask can be insufflated with air to increase the pressure within the foam. The compliance of the foam may be temperature sensitive. The foam will naturally tend to conform to the airway anatomy, but can be stiffened by insufflating air into foam within the plastic material of the mask, or ‘de-stiffened’ by withdrawing air from the foam within the plastic material of the mask. Thus, the improved mask component of this invention both naturally tends to conform to the airway and can also be manually adjusted.

### Advantageous Effects of Invention

The invention eliminates the problem of stagnant gas volume in breathing tubes. This problem can be especially pronounced in spontaneously breathing small patients with a fast and shallow respiratory pattern. Some patients, either because of medical condition or age, or a combination of both, breathe with a ventilatory pattern of fast and shallow breathing. That is, the tidal volume is relatively smaller and the respiratory rate is relatively faster. Because the tidal volumes are smaller, the stagnant volume of breathing tubes can be significant compared to tidal volume, such that with spontaneous ventilation it may be difficult (or even impossible) to maintain adequate ventilation during anesthesia such that adequate oxygenation and adequate elimination of carbon dioxide take place. This is especially important during anesthesia with the inhalational anesthetic agents. For example, nitrous oxide is known to increase respiratory rate and decrease tidal volume. The volatile anesthetic agent halothane causes rapid, shallow breathing. Similar ventilator effects are seen with the newer inhalational volatile anesthetic desflurane.

The stagnant volume within a laryngeal mask airway device is even more pronounced than with a corresponding endotracheal tube because in general the diameter of the breathing tube of a laryngeal mask airway device is larger than the diameter of an endotracheal tube for the same size patient. Thus, the volume of stagnant air in a laryngeal mask airway device will tend to be larger than the volume of stagnant air in an endotracheal tube. Laryngeal mask airway device use is increasingly significantly because it's ease of use and because it does not cause the trauma to the vocal cords that is often seen with endotracheal tube use. Also, laryngoscopy is not required. Laryngoscopy can be traumatic to the soft tissues in the larynx and often results in patient discomfort.

An embodiment of the invention described herein comprises an improved laryngeal mask component that provides for a naturally conforming yet adjustable anatomical fit, which is more likely to provide optimal ventilating conditions for a particular patient. This improved device may be used with the multi-lumen tube described herein, or with a single lumen tube described in the prior art.

Another advantage of this invention is a practical one. The multi-lumen breathing tube device, in addition to being used to separate the inhalation air from the exhalation air as previously described herein, may also be used with a y-piece or other connector not comprising a septum-like divider such that fresh gas flow may be inspired through both lumens and expired through both lumens. Thus, the invention described herein can act as a universal breathing tube where when interlocked with the divided connectors described herein separate inhalation air and exhalation are, but when used with non-divided connectors can function similarly to prior art breathing tubes. Thus, the present invention renders the prior art breathing tubes obsolete. For this reason, the inhalation lumen and exhalation lumen provide for a means to separate the inhalation air and the exhalation air for the length of the lumens, which may be caused to occur based on whether the inhalation air and exhalation air are separated in different compartments upon entering the multi-lumen breathing tube device due to the connectors and/or configurations described herein.

### DESCRIPTION OF DRAWINGS

The following drawings are not intended to limit the scope of the present invention in any way. Rather, they are used for illustrative purposes only. The invention is further determined by additional written descriptions described herein.

As discussed, FIG. 1 represents a typical circle breathing circuit used with many, if not most, anesthesia machines and mechanical ventilators in intensive care units. Because the circle circuit breathing system is universally used, it is important to develop a solution to the stagnant gas volume problem in breathing tubes in a way that is easily adapted to circle breathing circuits. 1 represents the elbow connector connecting the y-piece 2 to the inspiratory limb 3 and the expiratory limb 4 of the circle breathing circuit. 5 represents a one-way valve allowing for unidirectional flow in the inspiratory limb, and 6 represents a one-way valve allowing for unidirectional flow through the expiratory limb. The arrows represent flow of medical air. 7 represents a pressure relief valve whereby the air pressure within the circuit may be adjusted. 8 represents a reservoir air bag that can be manually squeezed to force air through the circle system. 9 represents a carbon dioxide absorbing system that cleanses the air in the expiratory limb from carbon dioxide before it is returned to the inspiratory limb. 10 represents an originating source of medical air into the circle circuit. In general there is a continuous flow of air through the system in the direction depicted by the arrows.

As discussed, FIG. 2A through FIG. 2D show the problem of stagnant gas volume in breathing tubes presently available in the marketplace. FIG. 2E through FIG. 2H represent the multi-lumen tube device described herein, and show how the present invention solves the problems of stagnant gas volume in breathing tubes by compartmentalizing the tube into separate channels for fresh inspiratory gas (new air) and expiratory gas, thus effectively moving the MDPFGF to a more desirable location; the MDPFGF is moved from 28 to 29. The vertical lines represent new air, and the horizontal lines represent expired air containing less oxygen and more carbon dioxide than new air.

FIG. 2A represents air flow through the y-piece and breathing tube of a breathing circuit prior to expiration of gas by the mammal and during inspiration; effectively the entire circuit contains only new air. 20 represents the inflow portion of the y-piece, and 22 represents the outflow portion of the y-piece. FIG. 2B represents expiration, demonstrating the mixing of new air with expired air above the MDPFGF at 28, and stagnant air below the MDPFGF at the end of expiration. FIG. 2C represents the beginning of subsequent inspiratory gas flow where new air is found above the MDPFGF at 28; at the beginning of this inspiration the tube below the MDPFGF at 28 is full of expired (stagnant) air; this stagnant air will be inhaled into the lung(s) during the inspiration. FIG. 2D demonstrates that with each subsequent exhalation a mixture of new air and stagnant air remains above the MDPFGF at 28, and that the tube below the MDPFGF contains stagnant air. This cycle then continues to repeat.

FIG. 2E demonstrates that when the compartmentalized breathing tube described herein is utilized, new air is delivered through one compartment 33. 31 represents a septum that separates the inflow compartment 33 of the breathing tube from the outflow compartment 35. FIG. 2F demonstrates that the invention has new air in the inflow compartment and expired air in the outflow compartment during expiration. This effectively moves the MDPFGF to 29. FIGS. 2G and 2H show that at all times through repeating cycles of inspiration and expiration new air is contained within in inflow compartment and expired air is contained in the outflow compartment.

FIG. 3 is a representation of an embodiment of the invention where the multi-lumen breathing tube device is a novel endotracheal tube (“ETT”). 25 is a flat plate portion of a connector that would connect the ETT to either an elbow or a Y-piece. An embodiment of the connector is viewed in greater detail in FIGS. 7 through 10. A septum 53 (analogous to septum 31 in FIGS. 2E through 2H) separates one lumen from another inside of the connector. 35 represents the proximal end of one side of the connector (e.g., the side containing the inspiratory lumen) and 39 represents the distal end of the same side. 37 represents the proximal end of the other side of the connector (e.g., the side containing the expiratory lumen) and 41 represents the distal end of the same side. 27 represents the multi-lumen breathing tube. 53 represents the septum within the breathing tube that separates the tube into an inspiratory lumen and an expiratory lumen. 49 represents a very small lumen contained within the outer wall of the tube 27, one end of this very small lumen communicating with an inflatable cuff 47 near the distal end of the tube, and the other end communicating with an outer tube 51 through which air may be injected to inflate the cuff 47. 43 represents the distal opening of the EU, and 45 represents the so-called “Murphy's eye” that would ventilate the right upper lobe of a patient's lungs. 29 represents the region where the MDPFGF is located. In this example, after the septum 53 ends the tube 27 narrows. The narrowed portion of the novel EU is similar in diameter size as a typical EU, thus allowing for easy passage of that portion of the EU through the vocal cords. This breathing tube device is generally curved, and it is being viewed looking into the concavity of the curve with the ETT bowed away from the viewer.

FIG. 4 is a general representation of an embodiment of the invention where the multi-lumen breathing tube device is a novel laryngeal mask airway device (“LMAD”). This view shows the top of the breathing tube: a cross section of the septum 55, and cross section of the proximal ends of the inspiratory and expiratory lumens (57 and 59) of the tube. 61 very generally represents the laryngeal mask portion of the LMAD. 63 represents the distal end of the lumen corresponding to 57, and 65 represents the distal end of the lumen corresponding to 59. 63 and 65 both communicate with the cavity of the laryngeal mask 61 such that inspiratory gas is directed into the lungs through the lumen on one side of the septum 55 (e.g., through 57 and 63), and expiratory gas is carried away from the lungs through the lumen on the other side of the septum 55 (e.g. through 59 and 65). 127 generally represents another embodiment of the novel multi-lumen breathing tube.

FIG. 5A is a general representation of a partially assembled LMAD interfacing with the connector and an elbow 64. (A more detailed embodiment of the invention's elbow is described in FIGS. 11 and 12). The flat plate portion of the connector is labeled 25. 127 represents the multi-lumen breathing tube. FIG. 5A is viewed from the side.

FIG. 5B is the same, except that the LMAD is assembled. 64 represents an elbow of the present invention. 25 represents the flat plate portion of the connector. 127 represents the novel breathing tube containing at least two separate lumen, one for inspiratory gas(es) and the other for expiratory gases. 61 generally represents the laryngeal mask potion of the LMAD.

FIG. 6 is a general representation of another embodiment of the invention where the multi-lumen breathing tube device is a novel LMAD. Here, the LMAD comprises a split of the breathing tube itself into a Y formation, where, for example, the corrugated tubing of the inspiratory limb of a circle breathing circuit 3 is attached to a single lumen connector 70, which provides a means for further connection to another type standard single lumen connector 72, which then connects to one lumen extending from the Y of the breathing tube 227 and ending at the distal most end of the lumen 63. Likewise, the corrugated tubing of the expiratory limb of a circle breathing circuit 4 is attached to a single lumen connector 70, which provides a means for further connection to another type standard single lumen connector 72, which then connects to the other lumen extending from the Y of the breathing tube 227 and ending at the distal most end of that lumen 65. Here, the MDPFGF 66 is found within the open end cavity of the laryngeal mask in a region in close proximity to the glottis opening and vocal cords when the LMAD is in situ after it is correctly inserted into a patient. (The flat plate portion of connector 72 is analogous to the flat plate portion 24 of FIG. 2A).

FIGS. 7 and 8 represent different external views of the novel connector. FIG. 8 indicates with arrows how the inspiratory and expiratory flows are kept separate from one another through the connector and how the connector provides for means of two unidirectional flows in opposite directions through the connector. For example connector portion 39 would be inserted into the proximal lumen opening 57 in FIG. 4. Similarly, connector portion 41 would be inserted into the proximal lumen opening 59 in FIG. 4. After this insertion, there would be one contiguous communication between the distal lumen opening 63, and 57 of FIG. 4 with the lumen openings 75 and 79 FIGS. 7 and 8 such that gas would flow continuously from opening 75 to opening 63. Likewise, there would be one contiguous communication between the distal lumen opening 65, and 59, of FIG. 4 with the lumen openings 77 and 81 of FIGS. 7 and 8 such that gas would flow continuously from opening 77 to opening 65. The proximal septum 55 as represented in FIG. 4 could be accepted into the distal crevice 85 of the novel connector. Thus, the connector would fit snuggly into the breathing tube thereby maintaining a means to keep the inspiratory and expiratory gas flows separate from one another.

FIG. 9 is representative of the connector in FIGS. 7 and 8 with the dashed lines representing the inner lumen walls of the connector in ‘see through view’. This is meant to show that the connector contains two distinct lumens 87 and 89 that are kept separate from one another throughout the inside of the connector. Similarly, FIG. 10 is representative of the same connector as viewed in FIG. 8 but with the dashed lines representing aspects of the connector hidden from the external view.

FIG. 11 is an external side view representation of a novel “elbow” connector 64. FIG. 12 is another external view of the same elbow 64. An elbow connector is also referred to as a “right angle” piece, but the novel piece will be referred to herein as an elbow. One end of the elbow in cross section shows the septum 100 and the lumens 101 and 102 fits onto and directly communicates with the proximal end of the novel connector of FIGS. 7 through 10, such the proximal connector regions 35 and 37 enter into lumens 101 and 102. The septum 100 in FIG. 11 is accepted into the crevice 83 as indicated in FIGS. 7 and 8. In this way the elbow 64 fits snuggly with the connector of FIGS. 7 and 8 on the connector's proximal end, and the connector of FIGS. 7 and 8 fits snuggly at its distal end with the breathing tube at its proximal end (e.g., the connector region of 39 in FIGS. 7 and 8 is accepted into lumen 57 of FIG. 4, the connector region 41 of FIGS. 7 and 8 is accepted into lumen 59 of FIG. 4, and septum 55 of FIG. 4 is accepted into crevice 85 of FIG. 7.). Thus one contiguous and separate lumen is formed from the proximal end of the elbow at 97 in FIGS. 11 and 12 to the distal most end of the breathing tube lumen at 63 in FIG. 4, and another contiguous and separate lumen is formed from the proximal end of the elbow at 99 in FIGS. 11 and 12 to the distal most end of the breathing tube lumen at 65. FIG. 11 also generally shows one of two sampling ports 103 and the port's cap 107. FIG. 12 shows representations of two sampling ports 103 and 105, with their respective caps 107 and 109.

FIG. 13 represents one embodiment of the Y-piece 110. Here the Y-piece is shown in relation to corrugated tubing making up the inspiratory and expiratory limbs of a circle breathing circuit (3 and 4, respectively, in FIG. 1). The top (most distal portion) of the septum 112 in FIG. 13 is accepted into the crevice 95 in FIG. 11. Regions of the elbow 91 and 93 in FIG. 11 are accepted into the lumen openings 114 and 116 in FIG. 13. The septum 118 of the Y-piece (FIGS. 13 and 14) separates the gas flow through openings 114 and 120 from the gas flow through openings 116 and 122, such that there are separate flows of gas through the Y-piece 110 as indicated by the arrows in FIG. 15.

FIGS. 16 and 17 represent a novel Y-piece 130 that is a preferred embodiment for a Y-piece of a circle breathing circuit. It does not have a stationary septum as indicated FIGS. 13, 14 and 15. Instead, Y-piece 130 has grooves 134 and an accepting unit comprising two walls, 136 and 138, and a floor 140. These grooves and accepting unit accept a removable septum that fits snuggly into the grooves and accepting unit such that the Y-piece 130, when fitted with the removable septum, functions like the Y-piece 110 indicated in FIG. 15.

FIGS. 18 and 19 represent a novel elbow 150 that comprises a “removable” septum 152. The septum is contained throughout the elbow such that there is a continuous lumen from lumen opening 154 to lumen opening 162, and another continuous lumen from lumen opening 156 to lumen opening 160. However the “removable septum” 152 extends out past the lumen openings 154 and 156. The novel elbow 150 also has an outer wall of a smaller diameter tube 151, and an outer wall of a larger diameter tube 163. Thus, the removable septum of FIGS. 18 and 19 may be accepted by the grooves 134 and accepting unit 136, 138 and 140 in FIGS. 16 and 17. This is represented in FIGS. 20 and 2 which show a side view of the novel elbow 150 and the novel Y-piece 130. FIG. 20 represents the elbow 150 and the Y-piece 130 prior to insertion of the removable septum 152 into the accepting unit 136, 138 and 140.

FIG. 21 represents an assembly after the elbow 150 is fitted into the Y-piece 130. The walls of the accepting unit 136 and 138 are superimposed upon one another in a side ‘see through’ view where dotted lines represent the structures inside of the assembly. The removable septum 152 of the elbow 150 is accepted between the walls of the accepting unit 136 and 138. This snuggly fit acceptance causes the Y-piece to be separated into two compartments or lumens, one in communication with elbow 150 lumens 154 and 160, and the other in communication with elbow 150 lumens 156 and 162. 165 represents the wall of the neck 132 of the Y-piece 130. 151 as a solid line in FIG. 21 represents the outer wall of the smaller diameter tube of the elbow 150. However, where 151 is represented by dashed lines in FIG. 21 it represents the interface between the outer wall of the smaller diameter tube of the elbow and the inner surface of the tube within the neck 132 of the Y-piece 130. The difference in width in the side view of FIG. 21 between the edge of the removable septum 152 and the outer wall of the smaller diameter tube 151 approximates the depth of the groove 134 (as seen in FIG. 16) into which the removable septum 152 slides.

FIG. 22 represents a novel connector 170 that allows for the novel breathing tubes of FIG. 3 or FIG. 4 to connect directly to a novel Y-piece, similar to the Y-piece 130 in FIGS. 16 and 17 but with modifications to the shape of the accepting unit, and without the necessity of grooves 134 (as in FIG. 16), without using the novel elbow 150. In FIGS. 22 and 23, the removable septum 172 has a rounded contour instead of being rectangular. Also, its edges (e.g. 174) do not stick out beyond the outer wall diameter 176 of the tube portion 194 above the plate portion 190 of the connector that is bisected by the removable septum 172. Also the edges 174 are rounded to conform to the outer wall diameter 176, which is approximately the diameter of the inner wall (e.g., 133 in FIG. 16) of the neck of the Y-piece (e.g., 132 in FIG. 16) such that the outer wall 176 be accepted into neck of the Y-piece (e.g., a Y-piece analogous to 132 in FIG. 16) so that the outer wall 176 of that portion of the connector will fit snuggly against the inner wall (e.g., an inner wall analogous to 133 in FIG. 16) of the neck. The portion of the connector 170 below the plate portion 190 (that portion comprising 184, 186 and 188) interfaces with the novel breathing tube as previously described.

FIG. 24 shows another view of the novel connector 170 demonstrating two separate lumen, each of which is continuous from one open end of the connector 180 within lumen wall 186 (represented by dashed lines) to the other end of the lumen 187. The dashed lines are the hidden view of the walls of each separate lumen within the connector. One set of dashed lines 186 represents one lumen, the set of dashed lines marked 188 represents the other lumen. The bold arrows in FIG. 25 represent separate gas flow in one direction through one of the connector's lumen, and gas flow in the opposite direction in the other of the connector's lumen.

FIG. 26 represents how the present invention may be incorporated into current technology. The LMAD here is adopted from U.S. Pat. No. 7,546,838 B2 (“838”) describing methods of manufacture of a LMAD. In FIG. 26 a novel breathing tube of the present invention 327 is substituted for the breathing tube taught in '838. The multi-lumen characteristic of the tube is represented by the tube's septum 31. This results in an improved LMAD with the functionality of the present invention when interfaced with the connectors taught herein.

FIG. 27 represents the incorporation of the present invention with another LMAD available in the marketplace. It represents a LMAD that includes a lumen that may communicate with the esophagus, which is referred to as a gastric channel. The newly improved LMAD 200 of FIG. 27 incorporates the present invention into a LMAD that has a gastric channel. 202 represents the proximal opening of the gastric channel. 204 represents the distal opening of the gastric channel. Note that the gastric channel does not communicate with the opening 208 of the laryngeal mask portion 206 of the LMAD. Here, the invention is represented as two tubes within the LMAD proper 200. One tube (or lumen) of the present invention has an opening 216 at its proximal end, which communicates with its opening 212 at its distal end. The distal end 212 communicates with the opening 208 in the laryngeal mask portion 206 of the LMAD. The other tube (or lumen) of the present invention has an opening 214 at its proximal end, which communicates with its opening 210 at its distal end. The distal end 210 communicates with the opening 208 in the laryngeal mask portion 206 of the LMAD. These are drawn as two separate tubes for simple illustration. Note that the 216-212 tube is larger in diameter and centered in the LMAD 200, whereas the 214-210 tube is smaller in diameter and is off the one side. Such an orientation of tubes (or lumen) would allow a secondary tube, such as a fiberoptic scope or an endotracheal tube, to be passed into the proximal end 216 of the tube through the distal end 212 in order to pass the secondary tube further distally such as through the vocal cords of a patient and into the patient's trachea. Nevertheless, without imposition of a secondary tube, the 216-212 and 214-210 tubes would be available to function such that one could provide for inspiratory gas flow and the other for expiratory in conformity with the present invention. If tubes (lumen) of different diameter are used, it is preferred that the larger diameter tube be used for expiratory flow so as to lessen the resistance to expiratory flow in relation to inspiratory flow, thus lessening the likelihood of the phenomenon of trapping ventilatory gas within the lung.

FIG. 28 represents an improvement of the LMAD described in U.S. Pat. No. 6,792,948 B2 (“'948”), indicating septum 31 as dividing the major lumen of '948 in half, providing for a multi-lumen breathing tube device as described in the present invention.

FIGS. 29 and 30 show an example of a non-divided breathing tube connector as generally referred to in paragraph [0030].

### DESCRIPTION OF EMBODIMENTS

The present invention is not limited to the aforementioned drawings. The present invention includes additional embodiments. The septums and/or connectors for the multi-tube breathing system form a baffle. A baffle is defined in Webster's New Twentieth Century Dictionary, Unabridged Second Edition, ISBN #0-529-04852-3, as “an obstructing device, as a wall or screen, to hold back or turn aside the flow of liquids, gases, etc.” The aforementioned septums and/or interlocking connectors have the function of a baffle in that they hold back or prevent the mixing of inspiratory and expiratory gases within the multi-lumen breathing tube as described. The baffle may be extended by adding another interlocking connector or piece to the baffle such that contiguity of the separate lumens remain intact.

The following definitions are used herein: 1) a septum is a type of divider; as used herein a divider may be a septum; 2) a divider may comprise a wall surrounding a lumen, or walls surrounding more than one lumen; 3) a baffle is a divider or series of dividers that direct air flow or gases to a desired pathway; 4) a lumen is a compartment or passage within a tube; 5) exhalation is synonymous with expiration; 6) inhalation is synonymous with inspiration; 7) the terms “distal” and “proximal” are used in relation to the inspiratory limb 3, and expiratory limb 4, of the circle breathing circuit depicted in FIG. 1; if point A is proximal to point B then point A is closer to the limbs than point B; if point B is distal to point A then point B is further away from the limbs than point A.

The baffle may take other forms within the scope of the present invention. For example, the y-portion configuration of the LMAD of FIG. 6 may be part of an endotracheal tube (“ETT”). Instead of a connector as depicted if FIG. 3, the top (or proximal) portion of the ETT may be configured with the y-portion device shown in FIG. 6. The baffle comprising the connector shown in FIGS. 7 through 10 may be configured differently providing it allows for an interface with an ETT or LMAD on one end and an element of a breathing circuit on the other end, such that inspiratory and expiratory gases are kept separated within the multi-lumen breathing tube device.

The term ‘multi-lumen’ means comprising at least two lumens. A breathing tube means a tube used for ventilation of the lung(s) of a mammal, more specifically a human. One embodiment of the multi-lumen breathing tube device comprises a LMAD as depicted in FIG. 4, or FIGS. 5A and 5B, or FIG. 6, or FIG. 26, or FIG. 27, or FIG. 28. Another embodiment of the multi-lumen breathing tube comprises a connector as shown in FIGS. 7 through 10, as the connector comprises at least two lumens, a baffle, and is used for ventilation of the lungs. Another embodiment of the multi-lumen breathing tube device comprises the elbow shown in FIGS. 11 and 12; it also comprises at least two lumens, a baffle, and is used for lung ventilation. Another embodiment of the multi-lumen breathing tube comprises the y-piece shown in FIGS. 13 through 15, as it comprises at least two lumens, a baffle, and is used for ventilating lungs. Another embodiment is the y-piece shown in FIGS. 16 and 17 as it too comprises at least two lumens, a baffle, and is used for lung ventilation.

Another embodiment of the invention is the elbow shown in FIGS. 18 through 21 which also comprises at least two lumens, a baffle, and is used for lung ventilation. Another embodiment of the multi-lumen breathing tube is the device shown in FIG. 21 comprising the elbow of FIGS. 18 through 20, and the y-piece of FIGS. 16 and 17. Another embodiment of the multi-lumen breathing tube is the connector shown in FIGS. 22 through 25, which also comprises at least two lumens, a baffle, and is used for lung ventilation. FIGS. 26, 27 and 28 show improvements over prior art LMADs where each LMAD comprises the multi-lumen breathing tube, each of which is an embodiment of the multi-lumen breathing tube device.

There are additional improvements that are embodiments of the multi-lumen breathing tube device, or simply improvements over prior art single lumen LMADs. One such embodiment is a laryngeal mask portion of a LMAD that comprises temperature sensitive memory foam, such as foam similar to the foam of a Tempur-Pedic mattress (see www.tempurpedic.com). Other such memory foams are available for manufacture of the laryngeal mask portion. The foam encompassing the perimeter surface of the laryngeal mask portion is cover by, or contained within, a thin compliant plastic material that is impermeable to air. When the LMAD is well placed in the airway as intended, the foam will tend to naturally conform to the airway structure of the glottis area. A communication with the foam by way of a tube, such as a tube that inflates the laryngeal portion of prior art LMADs, can accept air from an outside source such as a syringe. When air is injected through this tube to foam proper the foam will increase in volume and surface area, and when air is removed the foam will decrease in volume and surface area. This allows for the fit of the laryngeal mask portion containing compliant foam to be adjusted in the airway.

### INDUSTRIAL APPLICABILITY

The invention has industrial applicability in the field of medical arts. There is a long-felt need for an improved LMAD and/or ETT that is beneficial to small or medically compromised patients. LMADs and ETTs are sold in the marketplace and are widely used throughout the world.

### REFERENCE TO DEPOSITED BIOLOGICAL MATERIAL

No reference to deposited biological material is required.

### SEQUENCE LISTING FREE TEXT

A Sequence Listing is not applicable.

I claim by letters patent:

1. A breathing device for separating inhalation air and exhalation air when inserted to or distal to the supraglottic region of a mammal, comprising:
a. bidirectional airway tube comprising an inhalation lumen having a proximal end and a distal end, and a separate exhalation lumen having a proximal end and a distal end, and a length;
b. an inhalation lumen extending from the proximal end to a most distal point of fresh gas flow at the distal end;
c. an exhalation lumen extending from the proximal end to a distal end proximate to the most distal point of fresh gas flow;
d. wherein said inhalation lumen and said exhalation lumen provide for a means to separate the inhalation air and the exhalation air substantially the length of said bidirectional airway tube.

2. A breathing device as recited in claim 1, further comprising:
a proximal first connector, said first connector having a divider to direct fresh gas flow through the inhalation lumen of said first connector to the inhalation lumen of said bidirectional airway tube, and to direct expired gas through the exhalation lumen of said first connector to the exhalation lumen of said bidirectional airway tube.

3. A breathing device as recited in claim 2, further comprising:
a proximal second connector, said second connector having a divider that interfaces with said first connector to direct fresh gas flow through the inhalation lumen of said second connector to the inhalation lumen of said first connector, and to direct expired gas through the exhalation lumen of said second connector to the exhalation lumen of said first connector.

4. A breathing device as recited in claim 3, further comprising:
a Y-piece connector having a divider that interfaces with an other connector to direct fresh gas flow through the inhalation lumen of said Y-piece to the inhalation lumen of said other connector, and to direct expired gas through the exhalation lumen of said Y-piece to the inhalation lumen of the other connector.

5. A breathing device as recited in claim 4, further comprising:
a. a Y-piece connector having a partial divider that accepts and interlocks with a divider that extends from the other connector;
b. said other connector with a divider that extends from it, wherein the divider that extends from it is complementary in structure with said partial divider of said Y-piece such that when interlocked said partial divider of said Y-piece and the divider that extends from said other connector form a baffle that directs fresh gas flow through the inhalation lumen of said Y-piece to the inhalation lumen of said other connector, and directs expired gas to the exhalation lumen of said other connector to the exhalation lumen of said Y-piece.

6. A device as recited in claim 1, further comprising a laryngeal mask.

7. A device as recited in claim 6 wherein said laryngeal mask further comprises temperature sensitive memory foam.

8. A device as recited in claim 5, further comprising a circle breathing circuit.

9. A device as recited in claim 1, further comprising an inflatable endotracheal cuff.

10. A breathing tube connector, said breathing tube connector having a divider to direct fresh gas flow through the inhalation lumen of said breathing tube connector to the inhalation lumen of said bidirectional airway tube, and to direct expired gas through the exhalation lumen of said breathing tube connector to the exhalation lumen of said bidirectional airway tube.

11. An elbow connector for a breathing tube, said elbow connector having a divider to direct fresh gas flow through the inhalation lumen of said elbow connector to the inhalation lumen of said breathing tube connector, and to direct expired gas through the exhalation lumen of said elbow connector to the exhalation lumen of said breathing tube connector.

12. The elbow connector recited in claim 11, further comprising at least one gas sampling port.

13. The elbow connector recited in claim 12, wherein said sampling port further comprises a Luer lock attachment mechanism.

14. A Y-piece for a circle breathing circuit having a partial divider that accepts and interlocks with a complementary divider from an other connector, such that when interlocked said partial divider of said Y-piece and the divider from said other connector form a baffle that directs fresh gas flow through the inhalation lumen of said Y-piece to the inhalation lumen of said other connector, and directs expired gas to the exhalation lumen of said other connector to the exhalation lumen of said Y-piece.

15. An improved laryngeal mask airway device further comprising,
a. bidirectional airway tube comprising an inhalation lumen having a proximal end and a distal end, and a separate exhalation lumen having a proximal end and a distal end, and a length;
b. an inhalation lumen extending from the proximal end to a most distal point of fresh gas flow at the distal end;
c. an exhalation lumen extending from the proximal end to a distal end proximate to the most distal point of fresh gas flow;
d. wherein said inhalation lumen and said exhalation lumen provide for a means to separate the inhalation air and the exhalation air substantially the length of said bidirectional airway tube.

16. An improved endotracheal tube further comprising,
a. bidirectional airway tube comprising an inhalation lumen having a proximal end and a distal end, and a separate exhalation lumen having a proximal end and a distal end, and a length;
b. an inhalation lumen extending from the proximal end to a most distal point of fresh gas flow at the distal end;
c. an exhalation lumen extending from the proximal end to a distal end proximate to the most distal point of fresh gas flow;
d. wherein said inhalation lumen and said exhalation lumen provide for a means to separate the inhalation air and the exhalation air substantially the length of said bidirectional airway tube.

17. An improved laryngeal mask airway device further comprising, a laryngeal mask that further comprises temperature sensitive memory foam.
